(19) United States
(12) Patent Application Publication
(10) Pub. No.: US 2026/0241372 A1
(43) Pub. Date: Aug. 20, 2026

(54) CATALYST, METHOD FOR PRODUCING CATALYST, AND METHOD FOR PRODUCING ACRYLONITRILE

(71) Applicant: ASAHI KASEI KABUSHIKI KAISHA, Tokyo (JP)

(72) Inventors: Shota MIURA, Tokyo (JP); Shota AIKI, Tokyo (JP)

(73) Assignee: ASAHI KASEI KABUSHIKI KAISHA, Tokyo (JP)

(21) Appl. No.: 19/161,525

(22) PCT Filed: Feb. 27, 2024
(86) PCT No.: PCT/JP2024/007070

§ 371 Date: Sep. 2, 2025

(30) Foreign Application Priority Data
Mar. 2, 2023 (JP) 2023-032139
Mar. 2, 2023 (JP) 2023-032159

(51) Int. Cl. B01J 23/89; B01J 21/08; B01J 37/00; B01J 37/04; C07C 253/26; C07C 255/03; C07C 255/08

(52) U.S. Cl. CPC B01J 23/8946; B01J 21/08; B01J 37/0045; B01J 37/04; C07C 253/26; C07C 255/03; C07C 255/08

(57) ABSTRACT

Provided is an ammoxidation reaction catalyst comprising a support and a metal oxide supported on the support, wherein the support comprises silica; the metal oxide is represented by the following formula (1): Mo_{12-c}BiₐFe_{b}W_{c}Ce_{d}XₑY_{f}Z_{g}RbₕOᵢ . . . (1) wherein X to Z and a to i are as described in the present specification; an amount of the support is 46.0% by mass or less based on the mass of the ammoxidation reaction catalyst; a value of a+c+d calculated from the atomic ratios described in the formula (1) is 0.21 or more and 1.82 or less; and a value of d/(a+d) calculated from the atomic ratios described in the formula (1) is more than 0.50 and 0.90 or less.

### TECHNICAL FIELD

The present invention relates to a catalyst and a method for producing the same, and a method for producing acrylonitrile using the catalyst.

### BACKGROUND ART

Production methods through ammoxidation reaction which involves reacting propylene, ammonia, and molecular oxygen in the presence of a catalyst are known as methods for producing acrylonitrile. In this reaction, acetonitrile can be obtained as a by-product of acrylonitrile.

A number of oxide catalysts that are composed mainly of molybdenum, bismuth, and iron and are also supplemented with other metal elements have been proposed as catalysts for achieving favorable acrylonitrile yields in the reaction described above (see, for example, Patent Literature 1).

In order to secure high catalytic activity of such a catalyst, a metal oxide serving as an active site is generally supported in the state of fine particles on a support.

### CITATION LIST

### Patent Literature

Patent Literature 1: Japanese Patent No. 6342091

### SUMMARY OF INVENTION

### Technical Problem

In the ammoxidation reaction of propylene, a major product acrylonitrile as well as a by-product acetonitrile is industrially beneficial, and high-yield production of acrylonitrile and acetonitrile is demanded. Although the catalysts disclosed in Patent Literature 1 are largely improved in initial acrylonitrile yield and yield stability under long-term reaction conditions, this literature has no mention about an acetonitrile yield. Thus, any catalyst that sufficiently satisfies both the yields has not yet been obtained.

It is generally known about catalysts that under long-term reaction conditions, catalytic activity is reduced over time due to decrease in specific surface area of a catalyst associated with the sintering of a support or due to decrease in the number of active sites caused by the aggregation of metal oxide particles serving as active sites. Such reduced catalytic activity hinders the progression of conversion reaction of propylene and eventually induces time—dependent decrease in acrylonitrile yield. Although the catalysts disclosed in Patent Literature 1 are largely improved in initial acrylonitrile yield and yield stability under long-term reaction conditions, this literature has no mention about catalytic activity stability under long-term reaction conditions. Thus, performance that can achieve both of a high acrylonitrile yield and catalytic activity stability has not yet been satisfied.

The present invention has been made in light of the problems described above and provides a catalyst that enables acrylonitrile and acetonitrile to be produced at high yields under long-term reaction conditions without reducing the propylene activity of the catalyst in the production of acrylonitrile through the ammoxidation reaction of propylene, and a method for producing the same, and a method for producing acrylonitrile using the catalyst.

### Solution to Problem

The present inventors have completed the present invention by finding that the problems described above can be solved by using a catalyst having a metal oxide having specific composition and a specific amount of a silica support.

Specifically, the present invention is as follows.

[1]

An ammoxidation reaction catalyst comprising a support and a metal oxide supported on the support, wherein
the support comprises silica;
the metal oxide is represented by the following formula (1):

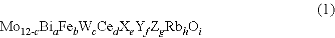

wherein
X is one or more elements selected from the group consisting of nickel, cobalt, magnesium, calcium, zinc, strontium, and barium,
Y is one or more elements selected from the group consisting of chromium, lanthanum, neodymium, yttrium, praseodymium, samarium, aluminum, gallium, and indium,
Z is one or more elements selected from the group consisting of potassium and cesium,
a to h satisfy the following relationships:

$$0.1 \leq a \leq 2. ,$$

$$0.1 \leq b \leq 3. ,$$

$$0.01 \leq c \leq 1. ,$$

$$0 < d \leq 3 \text{.0} ,$$

$$0 \leq e \leq 10 \text{.0} ,$$

$$0 \leq f \leq 3 \text{.0} ,$$

$$0 \leq g \leq 2. , and$$

$$0.1 \leq h \leq 0.2 ,$$

and
i is the number of oxygen atoms necessary for satisfying valence requirements of the other elements present;
an amount of the support is 46.0% by mass or less based on the mass of the ammoxidation reaction catalyst;
a value of a+c+d calculated from the atomic ratios described in the formula (1) is 0.21 or more and 1.82 or less; and
a value of d/(a+d) calculated from the atomic ratios described in the formula (1) is more than 0.50 and 0.90 or less.
   [2]

The ammoxidation reaction catalyst according to [1], wherein a value of c/d calculated from the atomic ratios described in the formula (1) is 0.0010 or more and 0.75 or less.

[3]

The ammoxidation reaction catalyst according to [1] or [2], wherein a value of c/h calculated from the atomic ratios described in the formula (1) is 0.050 or more and 3.5 or less.

[4]

The ammoxidation reaction catalyst according to any of [1] to [3], wherein
c described in the formula (1) is 0.01 or more and 0.55 or less;
the value of a+c+d calculated from the atomic ratios described in the formula (1) is 0.30 or more and 1.6 or less;
a value of c/d calculated from the atomic ratios described in the formula (1) is 0.07 or more and 0.75 or less;
a value of c/h calculated from the atomic ratios described in the formula (1) is 0.05 or more and 3.5 or less; and
the amount of the support is 20.0% by mass or more and 46.0% by mass or less based on the mass of the ammoxidation reaction catalyst.
   [5]

A method for producing the ammoxidation reaction catalyst according to any of [1] to [4], comprising:
a first step of preparing a starting material slurry;
a second step of spray-drying the starting material slurry to obtain dry particles, and
a third step of calcining the dry particles.
   [6]

A method for producing acrylonitrile and/or acetonitrile, comprising the step of reacting propylene, molecular oxygen, and ammonia in the presence of the ammoxidation reaction catalyst according to any of [1] to [4].

[7]

A method for producing (meth)acrylonitrile and/or acetonitrile, comprising the step of reacting isobutene or tertiary alcohol, molecular oxygen, and ammonia in the presence of the ammoxidation reaction catalyst according to any of [1] to [4].

### Advantageous Effect of Invention

Use of the ammoxidation reaction catalyst of the present invention enables acrylonitrile and acetonitrile to be produced at high yields over a long period without reducing the propylene activity of the catalyst in the production of acrylonitrile through the ammoxidation reaction of propylene.

### DESCRIPTION OF EMBODIMENTS

Next, the mode for carrying out the present invention will be specifically described. However, the present invention is not limited by the following embodiments and can be carried out through various changes or modifications made without departing from the spirit of the present invention.

### <Catalyst>

The ammoxidation reaction catalyst of the present embodiment is an ammoxidation reaction catalyst comprising a support and a metal oxide supported on the support, wherein
the support comprises silica;
the metal oxide is represented by the following formula (1):

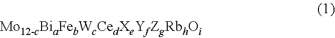

wherein
X is one or more elements selected from the group consisting of nickel, cobalt, magnesium, calcium, zinc, strontium, and barium,
Y is one or more elements selected from the group consisting of chromium, lanthanum, neodymium, yttrium, praseodymium, samarium, aluminum, gallium, and indium,
Z is one or more elements selected from the group consisting of potassium and cesium,
a to h satisfy the following relationships:

$$0.1 \leq a \leq 2. ,$$

$$0.1 \leq b \leq 3 \text{.0} ,$$

$$0.01 \leq c \leq 1. ( preferably 0.01 \leq c \leq 0.55 ) ,$$

$$0 < d \leq 3 \text{.0} ,$$

$$0 \leq e \leq 10 \text{.0} ,$$

$$0 \leq f \leq 3 \text{.0} ,$$

$$0 \leq g \leq 2. , and$$

$$0. 10 \leq h \leq 0.2 ,$$

and
i is the number of oxygen atoms necessary for satisfying valence requirements of the other elements present;
an amount of the support is 46.0% by mass or less based on the mass of the ammoxidation reaction catalyst;
a value of a+c+d calculated from the atomic ratios described in the formula (1) is 0.21 or more and 1.82 or less; and
a value of d/(a+d) calculated from the atomic ratios described in the formula (1) is more than 0.50 and 0.90 or less.

The ammoxidation reaction catalyst of the present embodiment thus configured enables acrylonitrile and acetonitrile to be produced at high yields over a long period without reducing the propylene activity of the catalyst in the production of acrylonitrile through the ammoxidation reaction of propylene.

### [Metal Oxide]

The metal oxide contained in the ammoxidation reaction catalyst of the present embodiment comprises molybdenum (Mo), bismuth (Bi), iron (Fe), tungsten (W), cerium (Ce), and rubidium (Rb) as essential components.

Molybdenum is a major element for forming the metal oxide and plays roles as an adsorption site for propylene and an activation site for ammonia.

In the formula (1), a represents the atomic ratio of bismuth to a total of 12 atoms of molybdenum and tungsten. Bismuth forms a complex oxide with molybdenum, iron, and cerium and becomes an element that forms an adsorption reaction site for propylene. A small content of bismuth cancels an adsorption reaction site for propylene and decreases the activity of propylene and the yield of acrylonitrile. Too large a content of bismuth increases degrading activity against propylene and tends to reduce selective reaction for acrylonitrile. From such a viewpoint, the suitable range of a is 0.1≤a≤2.0, preferably 0.15≤a≤1.0, more preferably 0.2≤a≤0.7.

In the formula (1), b represents the atomic ratio of iron to a total of 12 atoms of molybdenum and tungsten. Iron plays a role in taking up oxygen in a vapor phase into the catalyst and supplying this oxygen to a catalytic active site. Iron is presumed to have a function of promoting the reduction-oxidation ability of the catalyst at an adsorption reaction site for propylene. Upon ammoxidation of propylene, lattice oxygen in the catalyst is consumed so that the catalyst undergoes reductive action. When the reaction continues directly, oxygen in the catalyst is exhausted so that the ammoxidation reaction no longer progresses while the catalyst is deteriorated by reduction. Hence, a function of taking up oxygen in a vapor phase into the catalyst to suppress the deterioration of the catalyst by reduction is required, and iron is presumed to be responsible for this role. Iron with a small atomic ratio does not sufficiently work. A large atomic ratio of iron increases the oxidation ability of the catalyst and therefore increases degrading activity against propylene and decreases selectivity for acrylonitrile. From such a viewpoint, the suitable range of b is 0.1≤b≤3.0, preferably 0.5≤b≤2.5, more preferably 1.0≤b≤2.0.

In the formula (1), c represents the atomic ratio of tungsten to a total of 12 atoms of molybdenum and tungsten. Tungsten plays a role as an adsorption site for ammonia, as in molybdenum, and besides, is capable of substituting for the same site as the molybdenum site in crystals of the metal oxide formed in the catalyst. It is known that tungsten in the metal oxide tends to have stronger acidity than that of molybdenum. In the ammoxidation catalyst of the present embodiment as well, a reactive site where molybdenum and tungsten are complexed is presumed to exert favorable propylene conversion as compared with a reactive site of molybdenum alone. In short, it is considered that tungsten thus contained can increase the activity of the catalyst and can thus increase the acrylonitrile yield and acetonitrile yield of the catalyst. On the other hand, a tungsten-derived reactive site is also capable of promoting the degradation of produced acrylonitrile and acetonitrile. In short, an increased ratio of tungsten to molybdenum in the catalyst tends to improve propylene activity and however, tends to reduce selectivity for acrylonitrile and acetonitrile. The ammoxidation reaction is usually carried out at a temperature of 400 to 500° C. In such a high-temperature environment, molybdenum is gradually dispersed from the catalyst, causing induction of time-dependent reduction in catalyst performance. When the amount of tungsten in the catalyst is increased, the amount of molybdenum with respect to tungsten in the catalyst is decreased over time in association with the dispersion of molybdenum. Selectivity for acrylonitrile tends to be reduced over time, though propylene activity is improved over time. Thus, the value of c is an important factor for satisfying a high acrylonitrile yield and acetonitrile yield of the catalyst, and long-term stability of catalytic activity at the same time. From such a viewpoint, the suitable range of c is 0.01≤c≤1.0, preferably 0.01≤c≤0.70, more preferably 0.01≤c≤0.55, further preferably 0.01≤c≤0.50.

In the formula (1), d represents the atomic ratio of cerium to a total of 12 atoms of molybdenum and tungsten. Cerium plays a role in improving the structural stability of a complex oxide. Low thermal stability of the catalyst causes metal elements to move inside catalyst particles and might influence reduction in performance. A complex oxide having low structural stability particularly includes a complex metal oxide composed of bismuth and molybdenum (bismuth molybdate). Cerium has a function of improving the structural stability of such a complex metal oxide. The suitable range of d is 0≤d≤3.0, preferably 0.15≤d≤2.5, more preferably 0.2≤d≤2.0.

In the formula (1), e represents the atomic ratio of the element X to a total of 12 atoms of molybdenum and tungsten. The element X forms molybdate having a moderate lattice defect and plays a role in facilitating intra-bulk movement of oxygen. The element X is one or more elements selected from the group consisting of nickel, cobalt, magnesium, calcium, zinc, strontium, and barium, preferably one or more elements selected from the group consisting of nickel, cobalt, and magnesium, more preferably two or more elements selected from the group consisting of nickel, cobalt, and magnesium. The suitable range of e is 0≤e≤10.0, preferably 3.0≤e≤9.0, more preferably 5.0≤e≤8.5. When the element X is a combination of a plurality of elements, the value of e means the total atomic ratio of the plurality of elements.

In the formula (1), f represents the atomic ratio of the element Y to a total of 12 atoms of molybdenum and tungsten. The element Y, as in iron, is responsible for the uptake and supply of oxygen in the catalyst. The element Y is one or more elements selected from the group consisting of chromium, lanthanum, neodymium, yttrium, praseodymium, samarium, aluminum, gallium, and indium, preferably one or more elements selected from the group consisting of lanthanum, neodymium, praseodymium, and samarium. The suitable range of f is 0≤f≤3.0, preferably 0.2≤f≤2.0, more preferably 0.3≤f≤1.5. When the element Y is a combination of a plurality of elements, the value of f means the total atomic ratio of the plurality of elements.

In the formula (1), g represents the atomic ratio of the element Z to a total of 12 atoms of molybdenum and tungsten. The element Z is one or more elements selected from the group consisting of potassium and cesium. In the formula (1), h represents the atomic ratio of Rb to a total of 12 atoms of molybdenum and tungsten. The element Z and rubidium play a role in suppressing the degradation reaction of propylene or acrylonitrile and acetonitrile by covering an acid site present on the catalyst surface. These elements are also found to differ in selectivity for acrylonitrile. Cesium is more preferable than potassium, and rubidium is more preferable than cesium. The suitable range of g is 0≤g≤2.0, preferably 0.05≤g≤1.0.

The suitable range of h is 0.1≤h≤0.2, preferably 0.1≤h≤0.19, more preferably 0.1≤h≤0.18. When the element Z is a combination of a plurality of elements, the value of g means the total atomic ratio of the plurality of elements.

In the formula (1), I represents the atomic ratio of oxygen to a total of 12 atoms of molybdenum and tungsten and is the number of oxygen atoms necessary for satisfying valence requirements of the other elements present.

In the ammoxidation reaction catalyst of the present embodiment, the relationship between the atomic ratio a of bismuth and the atomic ratio d of cerium influences the yield of acrylonitrile and the long-term stability of catalytic activity. A small content of bismuth cancels an adsorption reaction site for propylene and decreases the activity of propylene and the yield of acrylonitrile. Cerium has, as mentioned above, a function of enhancing the structural stability of bismuth molybdate and however, has degrading activity against propylene. The presence of an excess of cerium reduces selectivity for acrylonitrile. The quantitative relationship between bismuth and cerium is important for maintaining an acrylonitrile yield and catalytic activity over a long period. When the value of d/(a+d) calculated from the atomic ratios is more than 0.50, long-term stability of catalyst performance is secured. When the value is 0.90 or less, decrease in acrylonitrile yield tends to be avoidable in each case. In short, the suitable range of the value of d/(a+d) is more than 0.50 and 0.90 or less, preferably 0.50 or more and 0.85 or less, more preferably 0.50 or more and 0.80 or less.

In the ammoxidation reaction catalyst of the present embodiment, the sum of bismuth, tungsten, and cerium in the catalyst is related to the amount of a main catalyst bismuth cerium molybdate produced as an active site in the catalyst. An increased sum of bismuth, tungsten, and cerium relatively increases the amount of bismuth molybdate containing tungsten and having a strong acid site and therefore induces the degradation of produced acrylonitrile and tends to decrease its yield.

Accordingly, the quantitative relationship among bismuth, tungsten, and cerium is important for increasing an acrylonitrile yield, and the sum a+c+d of a, c, and d shown in the formula (1) needs to fall within a specific range. In short, the suitable range of the value of a+c+d is 0.21≤a+c+d≤1.82, preferably 0.25≤a+c+d≤1.80, more preferably 0.3≤a+c+d≤1.60.

In the ammoxidation reaction catalyst of the present embodiment, cerium and tungsten in the catalyst are elements constituting a main catalyst crystal phase serving as a reactive site. The quantitative relationship between cerium and tungsten is important for forming a main catalyst crystal phase that exhibits high acrylonitrile selectivity, and the ratio c/d of c and d shown in the formula (1) needs to fall within a specific range. In short, the suitable range of the value of c/d is preferably 0.0010≤c/d≤0.75, more preferably 0.0030≤c/d≤0.75, further preferably 0.03≤c/d≤0.75, particularly preferably 0.07≤c/d≤0.75.

In the ammoxidation reaction catalyst of the present embodiment, a crystal phase containing tungsten in the catalyst serves as an acid site where propylene or a product is degraded, and rubidium is effective for covering a degradation site in the catalyst. The control of a degradation site in the catalyst is essential for maintaining catalytic activity and high yields of acrylonitrile and acetonitrile over a long period, and the quantitative relationship between tungsten and rubidium is important. In short, the ratio c/h of c and h shown in the formula (1) needs to fall within a specific range, and the suitable range of the value of c/h is preferably 0.050≤c/h≤3.5.

### [Support]

In the ammoxidation reaction catalyst of the present embodiment, the metal oxide is supported in the state of fine particles on a support. Examples of the support of the ammoxidation reaction catalyst include oxides such as silica, alumina, titania, and zirconia. Among them, silica is preferred because of small reduction in selectivity for acrylonitrile and excellent abrasion resistance and particle strength of the catalyst.

The starting material of silica for use as the support is not particularly limited, and a silica sol is preferred. Silica sols having different primary particle diameters may be used as a mixture. The primary particle size of the silica sol is not particularly limited and is preferably 1 nm or larger and 70 nm or smaller, more preferably 10 nm or larger and 50 nm or smaller.

### [Catalyst]

The catalyst of the present embodiment is suitably used in vapor phase catalytic ammoxidation reaction. The vapor phase catalytic ammoxidation reaction is reaction through which hydrocarbon, ammonia, and molecular oxygen are reacted in a vapor phase to produce unsaturated nitrile.

The ammoxidation reaction catalyst of the present embodiment can obtain higher catalytic activity in the production of acrylonitrile by setting its specific surface area to 10 m²/g or more, and can reduce the exposure of a degradation active site for propylene or acrylonitrile and suppress reduction in selectivity for acrylonitrile, by setting its specific surface area to 50 m²/g or less. The specific surface area of the catalyst is preferably 10 to 50 m²/g, more preferably 15 to 46 m²/g, further preferably 20 to 42 m²/g.

The proportion of the support in the ammoxidation reaction catalyst of the present embodiment is 46% by mass or less based on the mass of the catalyst. The lower limit value of the proportion is preferably 20% by mass, more preferably 30% by mass, further preferably 35% by mass. The upper limit value of the proportion is preferably 46% by mass, more preferably 44% by mass. When the amount of silica is 20% by mass or more, spherical particles necessary as a fluidized-bed catalyst tend to be easily formed. The smoothness of the particle surface is also improved, and both abrasion strength and crush strength are improved. On the other hand, when the amount of silica is 46% by mass or less, an acrylonitrile yield tends to be unlikely to be reduced. The proportion of the support is preferably 20 to 46% by mass, more preferably 30 to 44% by mass, further preferably 35 to 44% by mass, based on the mass of the catalyst.

The proportion of the metal oxide is preferably 54 to 80% by mass, more preferably 56 to 70% by mass, further preferably 56 to 65% by mass, based on the mass of the catalyst.

### <Method for Producing Catalyst>

One embodiment of the present invention relates to a method for producing a catalyst, comprising the steps of: providing a first mixed liquid containing a silica starting material, molybdenum, and tungsten; mixing the first mixed liquid with bismuth, iron, and cerium to obtain a second mixed liquid (starting material slurry); spray-drying the second mixed liquid to obtain particles; and calcining the particles to obtain a catalyst.

The production method of the present embodiment can produce the ammoxidation reaction catalyst described in the section <Catalyst>.

The starting material of each element source is not particularly limited and is preferably a salt soluble in water or nitric acid. Examples of the starting material of each element source of molybdenum, bismuth, iron, cerium, and tungsten include, but are not particularly limited to, ammonium salt, nitrate, hydrochloride, sulfate, organic acid salts, and inorganic salts. Particularly, the element sources of molybdenum and tungsten are preferably ammonium salt or an oxide, more preferably ammonium salt. The element sources of nickel, cobalt, magnesium, calcium, zinc, strontium, barium, chromium, yttrium, aluminum, gallium, indium, potassium, cesium, and rubidium are preferably their respective nitrates.

The silica starting material is preferably a silica sol. The silica concentration in the silica sol is preferably 10 to 50% by mass.

For the preparation of the starting material slurry, a carboxylic acid compound is preferably added to the starting material slurry. The carboxylic acid compound is a typical coordinating organic compound. This compound promotes high dispersion of metal components in the starting material slurry and tends to improve the acrylonitrile yield of the resulting catalyst. Examples of the carboxylic acid compound include, but are not particularly limited to, polyvalent carboxylic acid compounds such as oxalic acid, tartaric acid, succinic acid, malic acid, and citric acid. Oxalic acid and tartaric acid are preferred, and oxalic acid is more preferred. The silica starting material and the carboxylic acid compound are preferably mixed in advance.

A method for preparing the first mixed liquid is not particularly limited, and a silica sol, molybdenum, and tungsten can be mixed.

A method for preparing the second mixed liquid is not particularly limited, and the first mixed liquid can be mixed with bismuth, iron, and cerium. A further metal atom may be mixed therewith so as to attain desired catalyst composition. The details of the further metal atom and the catalyst composition are as described in the section <Catalyst>.

In the spray drying of the second mixed liquid, the entrance temperature of a spray dryer is preferably 100 to 400° C., more preferably 150 to 350° C., further preferably 200 to 300° C.

In the spray drying of the second mixed liquid, the exit temperature of the spray dryer is preferably 100 to 180° C., more preferably 100 to 150° C.

The calcination temperature of the particles obtained by spray drying is preferably 150 to 750° C., more preferably 300 to 700° C., further preferably 500 to 650° C.

### <Ammoxidation Reaction Catalyst>

The catalyst of the present embodiment is an ammoxidation reaction catalyst for producing acrylonitrile and/or acetonitrile by reacting propylene, ammonia, and molecular oxygen. The ammoxidation reaction catalyst of the present embodiment thus configured stably exhibits a high acrylonitrile yield and acetonitrile yield over a long period and besides, can suppress reduction in catalytic activity during reaction.

The catalyst of the present embodiment is an ammoxidation reaction catalyst for producing (meth)acrylonitrile and/or acetonitrile by reacting isobutene or tertiary alcohol, ammonia, and molecular oxygen. The ammoxidation reaction catalyst of the present embodiment thus configured stably exhibits a high (meth)acrylonitrile yield and acetonitrile yield over a long period and besides, can suppress reduction in catalytic activity during reaction.

### <Method for Producing Acrylonitrile>

One embodiment of the present invention relates to a method for producing acrylonitrile and/or acetonitrile, comprising the step of reacting propylene, ammonia, and molecular oxygen in the presence of the catalyst described in the section <Catalyst> to obtain acrylonitrile and/or acetonitrile.

One embodiment of the present invention relates to a method for producing (meth)acrylonitrile and/or acetonitrile, comprising the step of reacting isobutene or tertiary alcohol, ammonia, and molecular oxygen in the presence of the catalyst described in the section <Catalyst> to obtain (meth)acrylonitrile and/or acetonitrile.

The type of a reactor for carrying out the reaction is not particularly limited and is preferably a fluidized-bed reactor or a fixed-bed reactor, more preferably a fluidized-bed reactor.

In the reaction, the molar ratio among propylene (or isobutene or tertiary alcohol), ammonia, and air is preferably 1.0:0.8 to 2.5:7.0 to 14.0, more preferably 1.0:0.7 to 1.5:8.0 to 13.5.

The tertiary alcohol is preferably tert-butyl alcohol from the viewpoint of reactivity.

The reaction temperature is preferably 300 to 500° C., more preferably 400 to 500° C.

The reaction pressure is preferably 0.01 to 0.5 MPa, more preferably 0.05 to 0.3 MPa.

The contact time between the starting material gas and the catalyst is preferably 2 to 7 seconds, more preferably 3 to 6 seconds.

### EXAMPLES

Hereinafter, the present embodiment will be described in more detail with reference to Examples and Comparative Examples. However, the technical scope of the present invention is not limited by these examples. Catalyst composition described in each of Examples and Comparative Examples is the same value as that of composition of addition of each element. Examples of the analysis approach for the catalyst composition include fluorescent X-ray analysis apparatuses (XRF: X-ray fluorescence).

### [Acrylonitrile Yield and Acetonitrile Yield]

In accordance with the following evaluation method A, acrylonitrile was produced through the ammoxidation reaction of propylene using a catalyst obtained in each of Examples and Comparative Examples, and an acrylonitrile yield and an acetonitrile yield were calculated.

### [Evaluation Method A]

A Pyrex® glass tube with an inside diameter of 25 mm having 16 built-in 10-mesh wires at intervals of 1 cm was used as a reactor. The reactor was charged with 50 cc of the ammoxidation reaction catalyst. The reaction temperature was set to 430° C., and the reaction pressure at an entrance part of the reactor was set to 0.17 MPa. A mixed gas of propylene, ammonia, oxygen, and helium having 9% by volume of propylene was supplied thereto to carry out reaction. In this context, the volume ratio of ammonia to propylene was set such that a sulfuric acid intensity defined by the expression given below was 20±2 kg/T-AN. The volume ratio of oxygen to propylene was set such that an oxygen concentration in a gas at the exit of the reactor was 0.2%±0.02% by volume. A contact time defined by the expression given below was changed by changing the flow rate of the mixed gas. A propylene conversion rate defined by the expression given below was thereby set to 99.3±0.2%. In this context, the oxygen concentration in a gas at the exit of the reactor and the propylene conversion rate were obtained by sampling the gas at the exit of the reactor and analyzing the gas by gas chromatography. The yields of acrylonitrile and acetonitrile produced through this reaction were calculated according to the expression given below. The acrylonitrile yield and the acetonitrile yield were calculated as amounts of substances based on the number of carbon atoms in propylene. Therefore, in the expressions, coefficients corresponding to the numbers of carbon atoms in products were multiplied.

$$Sulfuric acid intensity ( kg / T - AN ) = \frac{\begin{matrix}Weight of sulfuric acid necessary for neutralizing \\ unreacted ammonia ( kg )\end{matrix}}{Production weight of acrylonitrike ( T )}$$

$$Contact time ( sec ) = \frac{Amount of catalyst ( cc )}{Mixed gas flow rate ( cc - NTP / sec )} \times \frac{273}{273 + Reaction temperature ( ° C . )} \times \frac{Reaction pressure ( MPa )}{0.1}$$

$$Propylene conversion rate ( \% ) = \frac{Consumed propylene ( mol )}{Supplied propylene ( mol )} \times 100$$

$$Acrylonitrile yield ( \% ) = \frac{Produced acrylonitrile ( mol )}{Supplied propylene ( mol )} \times 100$$

$$Acetonitrile yield ( \% ) = \frac{Produced acetonitrile ( mol )}{Supplied propylene ( mol )} \times \frac{2}{3} \times 100$$

Ammonium paramolybdate [(NH₄)₆Mo₇O₂₄·4H₂O] was added at 0.4 g per 50 cc of the catalyst to the catalyst every 300 hours from the start of reaction.

### [Catalytic Activity (Propylene Activity)]

Catalytic activity is an index that indicates the amplitude of propylene activity of the catalyst, and is calculated on the basis of a reaction rate calculated from the propylene conversion rate determined by the evaluation approach A.

In accordance with the following evaluation method B, acrylonitrile was produced through the ammoxidation reaction of propylene using a catalyst obtained in each of Examples and Comparative Examples, and catalytic activity was calculated.

### [Evaluation Method B]

A SUS316 reaction tube having an inside diameter of 10 mm was used as a reactor. The reactor was charged with 1 cc of the ammoxidation reaction catalyst. The reaction temperature was set to 440° C., and the reaction pressure at an entrance part of the reactor was set to a spontaneous pressure. A mixed gas of propylene/ammonia/oxygen/helium was supplied at 40 cc/sec (NTP-based) in terms of a total gas flow rate to carry out reaction. In this respect, the content of propylene in the mixed gas was set to 5.4% by volume, the propylene/ammonia/oxygen/water molar ratio was set to 1/1.2/1.89/1.85, and the flow rate of helium was set such that the total gas flow rate was 40 cc/sec (NTP-based). According to the expressions mentioned above, a contact time was calculated from the flow rate of the mixed gas, and a propylene conversion rate was calculated from the values of supplied propylene and consumed propylene. Catalytic activity was calculated according to the following expression using the obtained values.

$$Catalytic activity ( 1 / Hr ) = - 3600 / ( Contact time ( sec ) ) \times ln ( ( 100 - Propylene conversion rate ( \% ) ) / 100 )$$

wherein In represents a natural log.

The catalytic activity obtained by the evaluation method B was preferably 7.5 (10³/Hr) or more for a catalyst industrially used. A positive rate of change in activity after a lapse of 24 hours and after a lapse of 1000 hours from the start of reaction was determined as being more preferable.

### Example 1

A catalyst in which a metal oxide represented by the compositional formula Mo_{11.66}Bi_{0.34}Fe_{1.7}Ce_{0.68}W_{0.34}Co_{4.2}Ni_{3.3}Rb_{0.14} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 468.0 g of ammonium paramolybdate [(NH₄)₆Mo₇O₂₄·4H₂O] dissolved in 759.7 g of water and 35.1 g of an aqueous solution containing 15.0% by mass of ammonia were mixed and added to the mixed liquid. Further, a liquid of 19.2 g of ammonium metatungstate [(NH₄)₆H₂W₁₂O₄₀·4H₂O] dissolved in 100.0 g of water was added to the mixed liquid of the silica sols to obtain a first mixed liquid.

Next, 37.8 g of bismuth nitrate [Bi(NO₃)₃·5H₂O], 156.0 g of iron nitrate [Fe(NO₃)₃·9H₂O], 279.6 g of cobalt nitrate [Co(NO₃)₂·6H₂O], 220.2 g of nickel nitrate [Ni(NO₃)₂·6H₂O], 66.9 g of cerium nitrate [Ce(NO₃)₃·6H₂O], and 4.67 g of rubidium nitrate [RbNO₃] were dissolved in 393.1 g of an aqueous nitric acid solution having a concentration of 16.6% by mass, and the resulting liquid was added to the first mixed liquid and stirred at 40° C. for 1 hour to obtain a second mixed liquid.

Next, the second mixed liquid was spray-dried under conditions involving an entrance temperature of approximately 230° C. and an exit temperature of approximately 110° C. using a spraying apparatus with a dish-shaped rotor installed at the upper center of a dryer. In this respect, the number of rotations of the disc was set to 12500 rpm/min. The obtained dry particles were heated and denigrated at 200° C. for 5 minutes and further at 450° C. for 20 minutes after temperature increase at 2.5° C./min from 200° C. to 450° C. to obtain a denigrated powder. The obtained denigrated powder was calcined at 585° C. for 2 hours to obtain a catalyst. The obtained catalyst had a specific surface area of 36.0 m²/g.

In accordance with the evaluation method A mentioned above, acrylonitrile was produced through the ammoxidation reaction of propylene using the obtained catalyst. The acrylonitrile (in Table 1, represented by “AN”) yield after a lapse of 24 hours and after a lapse of 1000 hours from the start of reaction was 84.8% and 84.3%, respectively. Table 1 shows catalyst preparation conditions and performance evaluation results. The acetonitrile (in Table 1, represented by “MeCN”) yield after a lapse of 24 hours and after a lapse of 1000 hours from the start of reaction was 2.1% and 2.3%, respectively. Table 1 shows catalyst preparation conditions and performance evaluation results.

The reaction described above was carried out, and the propylene activity of the catalyst was evaluated by the following measurement method B using the catalyst after a lapse of 24 hours and after a lapse of 1000 hours from the start of reaction. The activity was 8.7 (10³/Hr) and 11.2 (10³/Hr), respectively. Performance evaluation results are shown in Table 1.

### Examples 2 to 5, Examples 7 to 10, Comparative Example 2, and Comparative Examples 4 to 6

Each catalyst was obtained by the same operation as in Example 1 in accordance with the conditions of Table 1. The performance evaluation of the catalyst was carried out by the same operation as in Example 1. Table 1 shows catalyst preparation conditions and performance evaluation results.

### Example 6

A catalyst in which a metal oxide having composition of Mo_{11.66}Bi_{0.34}Fe_{1.7}Ce_{0.68}W_{0.34}Ni_{5.0}Mg_{2.5}Rb_{0.14} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 483.8 g of ammonium paramolybdate [(NH₄)₆Mo₇O₂₄·4H₂O] dissolved in 788.8 g of water and 36.3 g of an aqueous solution containing 15.0% by mass of ammonia were mixed and added to the mixed liquid. Further, a liquid of 19.9 g of ammonium metatungstate [(NH₄)₆H₂W₁₂O₄₀·4H₂O] dissolved in 100.0 g of water was added to the mixed liquid of the silica sols to obtain a first mixed liquid.

Next, 39.0 g of bismuth nitrate [Bi(NO₃)₃·5H₂O], 161.2 g of iron nitrate [Fe(NO₃)₃·9H₂O], 344.9 g of nickel nitrate [Ni(NO₃)₂·6H₂O], 152.6 g of magnesium nitrate [Mg(NO₃)₂·6H₂O], 69.1 g of cerium nitrate [Ce(NO₃)₃·6H₂O], and 4.82 g of rubidium nitrate [RbNO₃] were dissolved in 393.1 g of an aqueous nitric acid solution having a concentration of 16.6% by mass, and the resulting liquid was added to the first mixed liquid and stirred at 40° C. for 1 hour to obtain a second mixed liquid.

Subsequent procedures were performed by the same operation as in Example 1 to obtain a catalyst. The performance evaluation of the catalyst was carried out by the same operation as in Example 1. Table 1 shows catalyst preparation conditions and performance evaluation results.

### Comparative Example 1

A catalyst was prepared and obtained by the same operation as in Example 1 in accordance with the conditions of Table 1 except that ammonium metatungstate [(NH₄)₆H₂W₁₂O₄₀·4H₂O] was not used. The performance evaluation of the catalyst was carried out by the same operation as in Example 1. Table 1 shows catalyst preparation conditions and performance evaluation results.

### Comparative Example 3

A catalyst was prepared and obtained by the same operation as in Example 6 in accordance with the conditions of Table 1 except that ammonium metatungstate [(NH₄)₆H₂W₁₂O₄₀·4H₂O] was not used. The performance evaluation of the catalyst was carried out by the same operation as in Example 1. Table 1 shows catalyst preparation conditions and performance evaluation results.

### Comparative Example 7

A catalyst in which a metal oxide having composition of Mo_{11.66}Bi_{0.34}Fe_{1.7}Ce_{0.68}W_{0.34}Co_{4.2}Ni_{3.3}K_{0.14}CS_{0.05} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 467.9 g of ammonium paramolybdate [(NH₄)₆Mo₇O₂₄·4H₂O] dissolved in 759.5 g of water and 35.1 g of an aqueous solution containing 15.0% by mass of ammonia were mixed and added to the mixed liquid. Further, a liquid of 19.2 g of ammonium metatungstate [(NH₄)₆H₂W₁₂O₄₀·4H₂O] dissolved in 100.0 g of water was added to the mixed liquid of the silica sols to obtain a first mixed liquid.

Next, 37.8 g of bismuth nitrate [Bi(NO₃)₃·5H₂O], 156.0 g of iron nitrate [Fe(NO₃)₃·9H₂O], 279.6 g of cobalt nitrate [Co(NO₃)₂·6H₂O], 220.1 g of nickel nitrate [Ni(NO₃)₂·6H₂O], 66.8 g of cerium nitrate [Ce(NO₃)₃·6H₂O], 3.20 g of potassium nitrate [KNO₃], and 2.20 g of cesium nitrate [CsNO₃] were dissolved in 393.1 g of an aqueous nitric acid solution having a concentration of 16.6% by mass, and the resulting liquid was added to the first mixed liquid and stirred at 40° C. for 1 hour to obtain a second mixed liquid.

Subsequent procedures were performed by the same operation as in Example 1 to obtain a catalyst. The performance evaluation of the catalyst was carried out by the same operation as in Example 1. Table 1 shows catalyst preparation conditions and performance evaluation results.

### Example 11

A catalyst in which a metal oxide represented by the compositional formula Mo_{11.70}Bi_{0.32}Fe_{1.7}Ce_{0.70}W_{0.30}Co_{4.0}Ni_{3.5}Rb_{0.15} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 470.3 g of ammonium paramolybdate [(NH₄)₆Mo₇O₂₄·4H₂O] dissolved in 761.0 g of water and 35.3 g of an aqueous solution containing 15.0% by mass of ammonia were mixed and added to the mixed liquid. Further, a liquid of 17.0 g of ammonium metatungstate [(NH₄)₆H₂W₁₂O₄₀·4H₂O] dissolved in 100.0 g of water was added to the mixed liquid of the silica sols to obtain a first mixed liquid.

Next, 35.6 g of bismuth nitrate [Bi(NO₃)₃·5H₂O], 156.2 g of iron nitrate [Fe(NO₃)₃·9H₂O], 266.7 g of cobalt nitrate [Co(NO₃)₂·6H₂O], 233.9 g of nickel nitrate [Ni(NO₃)₂·6H₂O], 68.9 g of cerium nitrate [Ce(NO₃)₃·6H₂O], and 5.01 g of rubidium nitrate [RbNO₃] were dissolved in 393.4 g of an aqueous nitric acid solution having a concentration of 16.6% by mass, and the resulting liquid was added to the first mixed liquid and stirred at 40° C. for 1 hour to obtain a second mixed liquid.

Next, the second mixed liquid was spray-dried under conditions involving an entrance temperature of approximately 230° C. and an exit temperature of approximately 110° C. using a spraying apparatus with a dish-shaped rotor installed at the upper center of a dryer. In this respect, the number of rotations of the disc was set to 12500 rpm/min. The obtained dry particles were heated and denigrated at 200° C. for 5 minutes and further at 450° C. for 20 minutes after temperature increase at 2.5° C./min from 200° C. to 450° C. to obtain a denigrated powder. The obtained denigrated powder was calcined at 585° C. for 2 hours to obtain a catalyst. The obtained catalyst had a specific surface area of 36.4 m²/g.

In accordance with the evaluation method A mentioned above, acrylonitrile was produced through the ammoxidation reaction of propylene using the obtained catalyst. The acrylonitrile (in Table 2, represented by “AN”) yield after a lapse of 24 hours and after a lapse of 1000 hours from the start of reaction was 84.9% and 84.4%, respectively. The acetonitrile (in Table 2, represented by “MeCN”) yield after a lapse of 24 hours and after a lapse of 1000 hours from the start of reaction was 2.1% and 2.4%, respectively. Table 2 shows catalyst preparation conditions and performance evaluation results.

### Examples 12 to 19, Examples 21 to 22, Comparative Example 9 to 10, Comparative Example 12, and Comparative Examples 14 to 15

Each catalyst was obtained by the same operation as in Example 11 in accordance with the conditions of Table 2. The performance evaluation of the catalyst was carried out by the same operation as in Example 11. Table 2 shows catalyst preparation conditions and performance evaluation results.

### Example 20

A catalyst in which a metal oxide having composition of Mo_{11.70}Bi_{0.32}Fe_{1.7}Ce_{0.70}W_{0.30}Ni_{4.0}Mg_{3.5}Rb_{0.15} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 492.8 g of ammonium paramolybdate [(NH₄)₆Mo₇O₂₄·4H₂O] dissolved in 802.2 g of water and 37.0 g of an aqueous solution containing 15.0% by mass of ammonia were mixed and added to the mixed liquid. Further, a liquid of 17.8 g of ammonium metatungstate [(NH₄)₆H₂W₁₂O₄₀·4H₂O] dissolved in 100.0 g of water was added to the mixed liquid of the silica sols to obtain a first mixed liquid.

Next, 37.3 g of bismuth nitrate [Bi(NO₃)₃·5H₂O], 163.7 g of iron nitrate [Fe(NO₃)₃·9H₂O], 280.1 g of nickel nitrate [Ni(NO₃)₂·6H₂O], 216.9 g of magnesium nitrate [Mg(NO₃)₂·6H₂O], 72.2 g of cerium nitrate [Ce(NO₃)₃·6H₂O], and 5.25 g of rubidium nitrate [RbNO₃] were dissolved in 401.8 g of an aqueous nitric acid solution having a concentration of 16.6% by mass, and the resulting liquid was added to the first mixed liquid and stirred at 40° C. for 1 hour to obtain a second mixed liquid.

Subsequent procedures were performed by the same operation as in Example 11 to obtain a catalyst. The performance evaluation of the catalyst was carried out by the same operation as in Example 11. Table 2 shows catalyst preparation conditions and performance evaluation results.

### Comparative Example 8

A catalyst was prepared and obtained by the same operation as in Example 11 in accordance with the conditions of Table 2 except that ammonium metatungstate [(NH₄)₆H₂W₁₂O₄₀·4H₂O] was not used. The performance evaluation of the catalyst was carried out by the same operation as in Example 11. Table 2 shows catalyst preparation conditions and performance evaluation results.

### Comparative Example 11

A catalyst was prepared and obtained by the same operation as in Example 20 in accordance with the conditions of Table 2 except that ammonium metatungstate [(NH₄)₆H₂W₁₂O₄₀·4H₂O] was not used. The performance evaluation of the catalyst was carried out by the same operation as in Example 11. Table 2 shows catalyst preparation conditions and performance evaluation results.

### Comparative Example 13

A catalyst in which a metal oxide having composition of Mo_{11.70}Bi_{0.32}Fe_{1.7}Ce_{0.70}W_{0.30}Co_{4.0}Ni_{3.5}K_{0.08}Cs_{0.07} was supported on 40% by mass of silica was produced by the following procedures.

1133.3 g of a first aqueous silica sol containing 30% by mass of silica having an average particle diameter of 12 nm for primary particles and 200.0 g of a second aqueous silica sol containing 30% by mass of silica having an average particle diameter of 41 nm for primary particles were mixed at a mass ratio of 85:15 to obtain a mixed liquid of the silica sols.

Next, 25.0 g of oxalic acid dihydrate dissolved in 287.5 g of water was added to the mixed liquid of the aqueous silica sols. Then, a liquid of 470.4 g of ammonium paramolybdate [(NH₄)₆Mo₇O₂₄·4H₂O] dissolved in 761.1 g of water and 35.3 g of an aqueous solution containing 15.0% by mass of ammonia were mixed and added to the mixed liquid. Further, a liquid of 17.0 g of ammonium metatungstate [(NH₄)₆H₂W₁₂O₄₀·4H₂O] dissolved in 100.0 g of water was added to the mixed liquid of the silica sols to obtain a first mixed liquid.

Next, 35.6 g of bismuth nitrate [Bi(NO₃)₃·5H₂O], 156.2 g of iron nitrate [Fe(NO₃)₃·9H₂O], 266.8 g of cobalt nitrate [Co(NO₃)₂·6H₂O], 233.9 g of nickel nitrate [Ni(NO₃)₂·6H₂O], 68.9 g of cerium nitrate [Ce(NO₃)₃·6H₂O], 1.83 g of potassium nitrate [KNO₃], and 3.09 g of cesium nitrate [CsNO₃] were dissolved in 393.4 g of an aqueous nitric acid solution having a concentration of 16.6% by mass, and the resulting liquid was added to the first mixed liquid and stirred at 40° C. for 1 hour to obtain a second mixed liquid.

Subsequent procedures were performed by the same operation as in Example 11 to obtain a catalyst. The performance evaluation of the catalyst was carried out by the same operation as in Example 11. Table 2 shows catalyst preparation conditions and performance evaluation results.

TABLE 1

| | | | | | | | | | | | | Silica | Elapsed |
|---|---|---|---|---|---|---|---|---|---|---|---|---|---|
| | | | | | | | | | | | | support | reaction |
| | Metal composition (atomic ratio) | | | | | | | | | | | (% by | time |
| | Mo | Bi | Fe | Ce | W | Co | Ni | Mg | K | Cs | Rb | mass) | (Hr) |
| Example 1 | 11.66 | 0.34 | 1.7 | 0.68 | 0.34 | 4.2 | 3.3 | 0 | 0 | 0 | 0.14 | 40 | 24 |
| | | | | | | | | | | | | | 1000 |
| Example 2 | 11.90 | 0.34 | 1.7 | 0.68 | 0.10 | 4.2 | 3.3 | 0 | 0 | 0 | 0.14 | 40 | 24 |
| | | | | | | | | | | | | | 1000 |
| Example 3 | 11.95 | 0.34 | 1.7 | 0.68 | 0.05 | 4.2 | 3.3 | 0 | 0 | 0 | 0.14 | 40 | 24 |
| | | | | | | | | | | | | | 1000 |
| Example 4 | 11.55 | 0.34 | 1.7 | 0.68 | 0.49 | 4.2 | 3.3 | 0 | 0 | 0 | 0.14 | 40 | 24 |
| | | | | | | | | | | | | | 1000 |
| Example 5 | 11.20 | 0.34 | 1.7 | 0.68 | 0.80 | 4.2 | 3.3 | 0 | 0 | 0 | 0.14 | 40 | 24 |
| | | | | | | | | | | | | | 1000 |
| Example 6 | 11.66 | 0.34 | 1.7 | 0.68 | 0.34 | 0 | 5.0 | 2.5 | 0 | 0 | 0.14 | 40 | 24 |
| | | | | | | | | | | | | | 1000 |
| Example 7 | 11.66 | 0.34 | 1.7 | 0.68 | 0.34 | 4.2 | 3.3 | 0 | 0 | 0 | 0.14 | 45 | 24 |
| | | | | | | | | | | | | | 1000 |
| Example 8 | 11.66 | 0.47 | 1.7 | 0.55 | 0.34 | 4.2 | 3.3 | 0 | 0 | 0 | 0.14 | 40 | 24 |
| | | | | | | | | | | | | | 1000 |
| Example 9 | 11.66 | 0.12 | 1.7 | 0.92 | 0.34 | 4.2 | 3.3 | 0 | 0 | 0 | 0.14 | 40 | 24 |
| | | | | | | | | | | | | | 1000 |
| Example 10 | 11.66 | 0.34 | 1.7 | 0.68 | 0.03 | 4.2 | 3.3 | 0 | 0 | 0 | 0.16 | 40 | 24 |
| | | | | | | | | | | | | | 1000 |
| Comparative | 12.00 | 0.34 | 1.7 | 0.68 | 0 | 4.2 | 3.3 | 0 | 0 | 0 | 0.14 | 40 | 24 |
| Example 1 | | | | | | | | | | | | | 1000 |
| Comparative | 10.90 | 0.34 | 1.7 | 0.68 | 1.10 | 4.2 | 3.3 | 0 | 0 | 0 | 0.14 | 40 | 24 |
| Example 2 | | | | | | | | | | | | | 1000 |
| Comparative | 12.00 | 0.54 | 1.7 | 0.48 | 0 | 0 | 5.0 | 2.5 | 0 | 0 | 0.14 | 40 | 24 |
| Example 3 | | | | | | | | | | | | | 1000 |
| Comparative | 11.66 | 0.54 | 1.7 | 0.48 | 0.34 | 4.2 | 3.3 | 0 | 0 | 0 | 0.14 | 40 | 24 |
| Example 4 | | | | | | | | | | | | | 1000 |
| Comparative | 11.66 | 0.34 | 1.7 | 0.68 | 0.34 | 4.2 | 3.3 | 0 | 0 | 0 | 0.14 | 50 | 24 |
| Example 5 | | | | | | | | | | | | | 1000 |
| Comparative | 11.66 | 0.08 | 1.7 | 0.96 | 0.34 | 4.2 | 3.3 | 0 | 0 | 0 | 0.14 | 40 | 24 |
| Example 6 | | | | | | | | | | | | | 1000 |
| Comparative | 11.66 | 0.34 | 1.7 | 0.68 | 0.34 | 4.2 | 3.3 | 0 | 0.14 | 0.05 | 0 | 40 | 24 |
| Example 7 | | | | | | | | | | | | | 1000 |

| | | | | | Rate of | | | | |
|---|---|---|---|---|---|---|---|---|---|
| | AN | Yield | MeCN | | change in | | | | |
| | yield | difference | yield | Activity | activity | a + | | d/ | |
| | (%) | (%) | (%) | (10³/Hr) | (%) | c + d | c/d | (a + d) | c/h |
| Example 1 | 84.8 | −0.5 | 2.1 | 8.7 | 30 | 1.36 | 0.50 | 0.67 | 2.4 |
| | 84.3 | | 2.3 | 11.2 | | | | | |
| Example 2 | 84.8 | −0.5 | 2.1 | 7.5 | 15 | 1.12 | 0.15 | 0.67 | 0.71 |
| | 84.3 | | 2.3 | 8.6 | | | | | |
| Example 3 | 84.7 | −0.4 | 2.0 | 7.7 | 8 | 1.07 | 0.074 | 0.67 | 0.36 |
| | 84.3 | | 2.1 | 8.4 | | | | | |
| Example 4 | 84.7 | −0.4 | 2.0 | 8.0 | 31 | 1.51 | 0.72 | 0.67 | 3.5 |
| | 84.3 | | 2.2 | 10.5 | | | | | |
| Example 5 | 84.6 | −0.6 | 2.0 | 7.8 | 26 | 1.82 | 1.2 | 0.67 | 5.7 |
| | 84.0 | | 2.1 | 9.8 | | | | | |
| Example 6 | 84.4 | −0.5 | 2.0 | 8.5 | 25 | 1.36 | 0.50 | 0.67 | 2.4 |
| | 83.9 | | 2.2 | 10.6 | | | | | |
| Example 7 | 84.7 | −0.4 | 2.1 | 8.9 | 24 | 1.36 | 0.50 | 0.67 | 2.4 |
| | 84.3 | | 2.2 | 11.0 | | | | | |
| Example 8 | 84.3 | −0.6 | 2.0 | 7.5 | 1 | 1.36 | 0.62 | 0.54 | 2.4 |
| | 83.7 | | 2.1 | 7.6 | | | | | |
| Example 9 | 84.1 | −0.2 | 2.0 | 7.3 | 11 | 1.38 | 0.37 | 0.88 | 2.4 |
| | 83.9 | | 2.0 | 8.1 | | | | | |
| Example 10 | 84.8 | −0.4 | 2.0 | 6.9 | 1 | 1.05 | 0.044 | 0.88 | 0.19 |
| | 84.4 | | 2.0 | 7.0 | | | | | |
| Comparative | 84.8 | −0.5 | 1.8 | 8.8 | −9 | 1.02 | 0 | 0.67 | 0 |
| Example 1 | 84.3 | | 1.8 | 7.9 | | | | | |
| Comparative | 84.6 | −1.6 | 1.8 | 7.9 | 31 | 2.12 | 1.6 | 0.67 | 7.9 |
| Example 2 | 83.0 | | 1.8 | 10.4 | | | | | |
| Comparative | 84.3 | −0.5 | 1.9 | 8.3 | −6 | 1.02 | 0 | 0.67 | 0 |
| Example 3 | 83.8 | | 1.9 | 7.8 | | | | | |
| Comparative | 84.3 | −0.8 | 1.9 | 7.9 | −10 | 1.36 | 0.71 | 0.47 | 2.4 |
| Example 4 | 83.5 | | 2.0 | 7.1 | | | | | |
| Comparative | 83.2 | −0.3 | 2.0 | 7.5 | 12 | 1.36 | 0.50 | 0.67 | 2.4 |
| Example 5 | 82.9 | | 2.1 | 8.4 | | | | | |
| Comparative | 82.8 | −0.6 | 2.0 | 7.1 | −11 | 1.38 | 0.35 | 0.92 | 2.4 |
| Example 6 | 82.2 | | 2.0 | 6.3 | | | | | |
| Comparative | 84.2 | −0.6 | 1.9 | 8.4 | 25 | 1.36 | 0.50 | 0.67 | — |
| Example 7 | 83.6 | | 1.9 | 10.5 | | | | | |

TABLE 2

| | | | | | | | | | | | | | Elapsed |
|---|---|---|---|---|---|---|---|---|---|---|---|---|---|
| | | | | | | | | | | | | Silica | reaction |
| | Metal composition (atomic ratio) | | | | | | | | | | | support | time |
| | Mo | Bi | Fe | Ce | W | Co | Ni | Mg | K | Cs | Rb | (Wt %) | (Hr) |
| Example 11 | 11.70 | 0.32 | 1.7 | 0.70 | 0.30 | 4.0 | 3.5 | 0 | 0 | 0 | 0.15 | 40 | 24 |
| | | | | | | | | | | | | | 1000 |
| Example 12 | 11.90 | 0.32 | 1.7 | 0.70 | 0.10 | 4.0 | 3.5 | 0 | 0 | 0 | 0.15 | 40 | 24 |
| | | | | | | | | | | | | | 1000 |
| Example 13 | 11.95 | 0.32 | 1.7 | 0.70 | 0.05 | 4.0 | 3.5 | 0 | 0 | 0 | 0.15 | 40 | 24 |
| | | | | | | | | | | | | | 1000 |
| Example 14 | 11.95 | 0.32 | 1.7 | 0.70 | 0.51 | 4.0 | 3.5 | 0 | 0 | 0 | 0.15 | 40 | 24 |
| | | | | | | | | | | | | | 1000 |
| Example 15 | 11.20 | 0.32 | 1.7 | 0.70 | 0.80 | 4.0 | 3.5 | 0 | 0 | 0 | 0.15 | 40 | 24 |
| | | | | | | | | | | | | | 1000 |
| Example 16 | 11.70 | 0.32 | 1.7 | 0.70 | 0.30 | 4.0 | 3.5 | 0 | 0 | 0 | 0.15 | 45 | 24 |
| | | | | | | | | | | | | | 1000 |
| Example 17 | 11.70 | 0.32 | 1.7 | 0.70 | 0.30 | 4.0 | 3.5 | 0 | 0 | 0 | 0.12 | 40 | 24 |
| | | | | | | | | | | | | | 1000 |
| Example 18 | 11.70 | 0.32 | 1.7 | 0.70 | 0.30 | 4.0 | 3.5 | 0 | 0 | 0 | 0.18 | 40 | 24 |
| | | | | | | | | | | | | | 1000 |
| Example 19 | 11.70 | 0.32 | 1.7 | 0.70 | 0.02 | 4.0 | 3.5 | 0 | 0 | 0 | 0.16 | 40 | 24 |
| | | | | | | | | | | | | | 1000 |
| Example 20 | 11.70 | 0.32 | 1.7 | 0.70 | 0.30 | 0 | 4.0 | 3.5 | 0 | 0 | 0.15 | 40 | 24 |
| | | | | | | | | | | | | | 1000 |
| Example 21 | 11.70 | 0.47 | 1.7 | 0.55 | 0.30 | 4.0 | 3.5 | 0 | 0 | 0 | 0.15 | 40 | 24 |
| | | | | | | | | | | | | | 1000 |
| Example 22 | 11.70 | 0.12 | 1.7 | 0.92 | 0.30 | 4.0 | 3.5 | 0 | 0 | 0 | 0.15 | 40 | 24 |
| | | | | | | | | | | | | | 1000 |
| Comparative | 12.00 | 0.32 | 1.7 | 0.70 | 0 | 4.0 | 3.5 | 0 | 0 | 0 | 0.15 | 40 | 24 |
| Example 8 | | | | | | | | | | | | | 1000 |
| Comparative | 10.90 | 0.32 | 1.7 | 0.70 | 1.10 | 4.0 | 3.5 | 0 | 0 | 0 | 0.15 | 40 | 24 |
| Example 9 | | | | | | | | | | | | | 1000 |
| Comparative | 10.90 | 0.50 | 1.7 | 0.75 | 0.60 | 4.0 | 3.5 | 0 | 0 | 0 | 0.15 | 40 | 24 |
| Example 10 | | | | | | | | | | | | | 1000 |
| Comparative | 12.00 | 0.32 | 1.7 | 0.70 | 0 | 0 | 4.0 | 3.5 | 0 | 0 | 0.15 | 40 | 24 |
| Example 11 | | | | | | | | | | | | | 1000 |
| Comparative | 11.70 | 0.32 | 1.7 | 0.70 | 0.30 | 4.0 | 3.5 | 0 | 0 | 0 | 0.15 | 50 | 24 |
| Example 12 | | | | | | | | | | | | | 1000 |
| Comparative | 11.70 | 0.32 | 1.7 | 0.70 | 0.30 | 4.0 | 3.5 | 0 | 0.08 | 0.07 | 0 | 40 | 24 |
| Example 13 | | | | | | | | | | | | | 1000 |
| Comparative | 11.70 | 0.32 | 1.7 | 0.70 | 0.30 | 4.0 | 3.5 | 0 | 0 | 0 | 0.08 | 40 | 24 |
| Example 14 | | | | | | | | | | | | | 1000 |
| Comparative | 11.70 | 0.32 | 1.7 | 0.70 | 0.30 | 4.0 | 3.5 | 0 | 0 | 0 | 0.22 | 40 | 24 |
| Example 15 | | | | | | | | | | | | | 1000 |

| | | | | | Rate of | | | | |
|---|---|---|---|---|---|---|---|---|---|
| | AN | Yield | MeCN | | change in | | | | |
| | yield | difference | yield | Activity | activity | a + | | d/ | |
| | (%) | (%) | (%) | (10³/Hr) | (%) | c + d | c/d | (a + d) | c/h |
| Example 11 | 84.9 | −0.5 | 2.1 | 8.4 | 25 | 1.32 | 0.43 | 0.69 | 2.0 |
| | 84.4 | | 2.4 | 10.5 | | | | | |
| Example 12 | 84.8 | −0.5 | 2.1 | 7.4 | 14 | 1.12 | 0.14 | 0.69 | 0.67 |
| | 84.3 | | 2.3 | 8.4 | | | | | |
| Example 13 | 84.7 | −0.4 | 2.0 | 7.5 | 7 | 1.07 | 0.071 | 0.69 | 0.33 |
| | 84.3 | | 2.2 | 8.0 | | | | | |
| Example 14 | 84.6 | −0.4 | 2.0 | 7.8 | 30 | 1.53 | 0.73 | 0.69 | 3.4 |
| | 84.2 | | 2.1 | 10.1 | | | | | |
| Example 15 | 84.6 | −0.7 | 2.1 | 7.5 | 28 | 1.82 | 1.1 | 0.69 | 5.3 |
| | 83.9 | | 2.2 | 9.6 | | | | | |
| Example 16 | 84.7 | −0.4 | 2.0 | 7.9 | 25 | 1.32 | 0.43 | 0.69 | 2.0 |
| | 84.3 | | 2.2 | 9.8 | | | | | |
| Example 17 | 84.6 | −0.2 | 2.1 | 8.9 | 22 | 1.32 | 0.43 | 0.69 | 2.5 |
| | 84.4 | | 2.2 | 10.9 | | | | | |
| Example 18 | 84.9 | −0.5 | 2.0 | 7.8 | 27 | 1.32 | 0.43 | 0.69 | 1.7 |
| | 84.4 | | 2.1 | 9.9 | | | | | |
| Example 19 | 84.9 | −0.5 | 2.0 | 8.6 | 2 | 1.04 | 0.03 | 0.69 | 0.13 |
| | 84.3 | | 2.1 | 8.7 | | | | | |
| Example 20 | 84.4 | −0.5 | 2.1 | 8.1 | 26 | 1.32 | 0.43 | 0.69 | 2.0 |
| | 83.9 | | 2.3 | 10.1 | | | | | |
| Example 21 | 84.2 | −0.6 | 2.1 | 7.3 | 3 | 1.32 | 0.55 | 0.54 | 2.0 |
| | 83.6 | | 2.2 | 7.5 | | | | | |
| Example 22 | 84.1 | −0.2 | 2.0 | 7.2 | 10 | 1.34 | 0.33 | 0.88 | 2.0 |
| | 83.9 | | 2.2 | 7.9 | | | | | |
| Comparative | 84.8 | −0.3 | 1.8 | 8.3 | −11 | 1.02 | 0 | 0.69 | 0 |
| Example 8 | 84.4 | | 1.8 | 7.4 | | | | | |
| Comparative | 84.6 | −1.6 | 1.8 | 7.7 | 33 | 2.12 | 1.6 | 0.69 | 7.3 |
| Example 9 | 83.0 | | 1.8 | 10.2 | | | | | |
| Comparative | 83.8 | −0.7 | 1.8 | 7.0 | 20 | 1.85 | 0.80 | 0.60 | 4.0 |
| Example 10 | 83.1 | | 1.9 | 8.4 | | | | | |
| Comparative | 84.2 | −0.5 | 1.8 | 8.0 | −8 | 1.02 | 0 | 0.69 | 0 |
| Example 11 | 83.7 | | 1.8 | 7.3 | | | | | |
| Comparative | 83.6 | −0.3 | 2.0 | 8.4 | 10 | 1.32 | 0.43 | 0.69 | 2.0 |
| Example 12 | 83.4 | | 2.1 | 9.2 | | | | | |
| Comparative | 84.2 | −0.6 | 1.9 | 8.1 | 24 | 1.32 | 0.43 | 0.69 | — |
| Example 13 | 83.6 | | 2.0 | 10.0 | | | | | |
| Comparative | 83.4 | −0.8 | 1.9 | 9.3 | 23 | 1.32 | 0.43 | 0.69 | 3.8 |
| Example 14 | 82.6 | | 2.0 | 11.5 | | | | | |
| Comparative | 84.4 | −0.8 | 1.8 | 6.1 | 23 | 1.32 | 0.43 | 0.69 | 1.4 |
| Example 15 | 83.6 | | 1.9 | 7.6 | | | | | |

1-7. (canceled)

8. An oxidation reaction catalyst comprising a support and a metal oxide supported on the support, wherein
the support comprises silica;
the metal oxide is represented by the following formula (1): 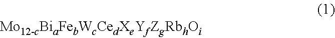
wherein
X is one or more elements selected from the group consisting of nickel, cobalt, magnesium, calcium, zinc, strontium, and barium,
Y is one or more elements selected from the group consisting of chromium, lanthanum, neodymium, yttrium, praseodymium, samarium, aluminum, gallium, and indium,
Z is one or more elements selected from the group consisting of potassium and cesium,
a to h satisfy the following relationships:
$$0.1 \leq a \leq 2. ,$$
$$0.1 \leq b \leq 3. ,$$
$$0.01 \leq c \leq 1. ,$$
$$0 < d \leq 3. ,$$
$$0 \leq e \leq 10 \text{.0} ,$$
$$0 \leq f \leq 3. ,$$
$$0 \leq g \leq 2. , and$$
$$0.1 \leq h \leq 0.2 ,$$
and
i is the number of oxygen atoms necessary for satisfying valence requirements of the other elements present;
an amount of the support is 46.0% by mass or less based on the mass of the oxidation reaction catalyst;
a value of a+c+d calculated from the atomic ratios described in the formula (1) is 0.21 or more and 1.82 or less; and
a value of d/(a+d) calculated from the atomic ratios described in the formula (1) is more than 0.50 and 0.90 or less.

9. The oxidation reaction catalyst according to claim 8, wherein the oxidation reaction catalyst is an ammoxidation reaction catalyst.

10. The oxidation reaction catalyst according to claim 8, wherein the oxidation reaction catalyst is a vapor phase catalytic oxidation reaction catalyst for reacting a hydrocarbon and molecular oxygen in a vapor phase.

11. The oxidation reaction catalyst according to claim 8, wherein a value of c/d calculated from the atomic ratios described in the formula (1) is 0.0010 or more and 0.75 or less.

12. The oxidation reaction catalyst according to claim 8, wherein a value of c/h calculated from the atomic ratios described in the formula (1) is 0.050 or more and 3.5 or less.

13. The oxidation reaction catalyst according to claim 8, wherein
c described in the formula (1) is 0.01 or more and 0.55 or less;
the value of a+c+d calculated from the atomic ratios described in the formula (1) is 0.30 or more and 1.6 or less;
a value of c/d calculated from the atomic ratios described in the formula (1) is 0.07 or more and 0.75 or less;
a value of c/h calculated from the atomic ratios described in the formula (1) is 0.05 or more and 3.5 or less; and
the amount of the support is 20.0% by mass or more and 46.0% by mass or less based on the mass of the oxidation reaction catalyst.

14. A method for producing the oxidation reaction catalyst according to claim 8, comprising:
a first step of preparing a starting material slurry;
a second step of spray-drying the starting material slurry to obtain dry particles; and
a third step of calcining the dry particles.

15. A method for producing acrylonitrile and/or acetonitrile, comprising the step of reacting propylene, molecular oxygen, and ammonia in the presence of the oxidation reaction catalyst according to claim 8.

16. A method for producing (meth)acrylonitrile and/or acetonitrile, comprising the step of reacting isobutene or tertiary alcohol, molecular oxygen, and ammonia in the presence of the oxidation reaction catalyst according to claim 8.

17. The oxidation reaction catalyst according to claim 11, wherein a value of c/h calculated from the atomic ratios described in the formula (1) is 0.050 or more and 3.5 or less.
